(19) United States
(12) Patent Application Publication
(10) Pub. No.: US 2012/0003335 A1
(43) Pub. Date: Jan. 5, 2012

(54) CONSTITUTIVE ANDROSTANE RECEPTOR (CAR) AS A THERAPEUTIC TARGET FOR OBESITY AND TYPE TWO DIABETES

(75) Inventors: Wen Xie, Pittsburgh, PA (US); Jie Gao, Pittsburgh, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 13/129,711

(22) PCT Filed: Nov. 18, 2009
(86) PCT No.: PCT/US09/64899

§ 371 Date: Sep. 19, 2011

Related U.S. Application Data
(60) Provisional application No. 61/115,764, filed on Nov. 18, 2008.

(51) Int. Cl. A61K 36/282; A61K 31/515; A61K 31/37; A61P 3/10; A61K 31/429; A61K 31/7088; A61K 31/421; A61P 3/04; A61K 31/444; A61K 31/497

(52) U.S. Cl. 424/740; 514/335; 514/270; 514/457; 514/255.05; 514/368; 514/44 R; 514/374

(57) ABSTRACT

The invention provides a method of controlling obesity or type two diabetes in a human. In accordance with the inventive method, the constitutive androstane receptor (CAR) is agonized within the human, which effectively controls obesity or type two diabetes.

### CROSS-REFERENCE TO RELATED APPLICATIONS

This application claims priority from U.S. Provisional Patent application 61/115,764, filed on Nov. 18, 2008, the contents of which are incorported herein in their entirety.

### STATEMENT REGARDING FEDERALLY SPONSORED RESEARCH AND DEVELOPMENT

This invention was made with Government support under Grant Number ES012479 awarded by the National Institute of Environmental Health Sciences and CA107011 awarded by the National Cancer Institute. The Government has certain rights in this invention.

### BRIEF SUMMARY OF THE INVENTION

The invention provides a method of controlling obesity or type two diabetes in a mammalian patient or subject. In accordance with the inventive method, the constitutive androstane receptor (CAR) is agonized within the patient, which effectively controls obesity or type two diabetes.

### BRIEF DESCRIPTION OF THE SEVERAL VIEWS OF THE DRAWING(S)

FIG. 1 (1A-1G) present data showing that the inventive method can control high fat diet-induced obesity and obesity in the genetically predisposed ob/ob mice.

FIG. 2 (2A-2D) present data showing that the inventive method can control high fat diet-induced obesity.

FIG. 3 (3A-3D) presents data showing that the inventive method can control type two diabetes.

FIG. 4 presents data showing that the inventive method activates Cyp2b10 gene expression.

FIG. 5 (5A-5F) presents data showing that the inventive method can inhibit lipogenesis, hepatic VLDL secretion, and hepatic gluconeogenesis.

FIG. 6 (6A-6E) presents data showing that the inventive method suppresses lipogenic and gluconeogenic gene expression in a CAR-dependent manner.

FIG. 7 (7A-7G) presents data showing that the inventive method can increase BAT energy expenditure.

FIG. 8 (8A-8C) presents data showing that the inventive method can decrease leptin production and inhibit food intake.

FIG. 9 presents data showing the expression of PCGla, G6P and PEPCK in wild wtpy and CAR-null mice.

FIG. 10 presents data showing that overexpression of CAR can lower blood glucose in fed and fasting wild type mice. Blood glucose levels are reported as mg/dl.

### DETAILED DESCRIPTION OF THE INVENTION

The invention provides a method of controlling obesity or type two diabetes in a patient or subject. The patient or subject can be any mammalian species (such as a laboratory animal (mouse, rat, monkey or ape), livestock (horse, cow, pig, goat), pet (cat or dog), or zoologically important mammal (e.g., large cat, ungulate, panda, etc.). More typically, the patient or subject is human. In accordance with the inventive method, the constitutive androstane receptor (CAR) is agonized within the patient or subject, which effectively controls obesity or type two diabetes.

In accordance with the inventive method, the diabetes or obesity is controlled if symptoms of the disease are reduced. Obesity is controlled by the inventive method if a patient or subject suffering from obesity loses weight. Some obese patient or subjects having undergone or currently undergoing treatment in accordance with the inventive method may lose sufficient weight to no longer be considered obese; however, other patients or subjects having undergone or currently undergoing treatment in accordance with the inventive method, while losing weight might nonetheless still be considered obese. Diabetes is controlled by the inventive method if the blood glucose level of a patient or subject having undergone or currently undergoing treatment in accordance with the inventive method moves closer to a nondiabetic level than prior to the treatment in accordance with the inventive method. Ideally for humans, this means glucose levels between 70 mg/dl and 130 mg/dl before meals, and less than 180 mg/dl two hours after starting a meal, with a glycated hemoglobin level less than 7 percent. However, the inventive method need not result in this ideal condition for it to be effective.

It should be understood that the inventive method can be employed adjunctively with other therapies for diabetes and/or obesity.

In one embodiment, CAR is agonized within the patient or subject by administering to the patient or subject a CAR agonist (or combination thereof) in an amount and at a location sufficient to agonize CAR within the patient or subject. Any suitable CAR agonist(s) can be employed. Examples of these include 1,4-bis[2-(3,5 dichloropyridyloxy)] benzene (TCPOBOP), Phenobarbital, 6-(4-chlorophenyl)imidazo[ 2,1-b][1,3]thiazole-5-carbaldehyde O-(3,4-dichlorobenzyl)oxime (CITCO), 6,7-Dimethylesculetin, oltipraz (OPZ), a pharmaceutically acceptable salt or hydrate of any thereof, and a combination of 2 or more thereof. It should be noted that TCPOBOP is not preferred for use in human patients as it is believed to be a mouse-specific CAR agonist.

In another embodiment, the CAR is agonized by administering a decoction of Artemisia capillaries to the patient or subject. While any suitable varietal can be employed, an exemplary Artemisia capillaries varietal is Yin Chin. Also, while any suitable decoction can be employed, an exemplary decoction is Yin Zhi Huang. The decoction can be administered once or several times daily, and it can be administered either with meals or alone.

In another embodiment, CAR can be agonized by increasing the expression of a CAR gene within the patient or subject, particularly in the liver. For example, CAR expression can be increased by introducing a genetic construct suitable for expressing CAR (e.g., a CAR expression vector comprising a CAR-encoding sequence under the control of a suitable promoter) into the patient or subject, such as within the liver of the subject. Within the patient or subject, thus, the CAR sequence is expressed from the vector such that the production of CAR is increased within the patient.

Preferably, the inventive method involving agonizing CAR is achieved without agonizing a peroxisome proliferator-activated receptor (PPAR) within the patient or subject. The PPAR can be, for example, PPARα, PPARβ/δ, PPARγ or a combination of two or more thereof. In certain applications, the inventive method can comprise antagonizing a PPAR within the patient or subject. The PPAR can, for example, be antagonized by administering to the patient or subject a PPAR antagonist (or combination of PPAR antagonists) in an amount and at a location to antagonize the PPAR within the patient or subject. Any suitable PPAR antagonist(s) can be employed, an exemplary one being [(2S)-2-[[(1Z)-1-Methyl-3-oxo-3-[4-(trifluoromethyl)phe nyl]-1-propenyl]amino]-3-[4-[2-(5-methyl-2-phenyl-4-oxa zolyl)ethoxy]phenyl]propyl]-carbamic acid ethyl ester (available as GW 6471 from Tocris Bioscience).

The CAR agonist(s) and, if employed, the PPAR antagonist(s) can be administered to the patient or subject at any suitable dose to achieve control of the diabetes and/or obesity. The appropriate dose can vary depending on the weight of the patient or subject, route of delivery, and severity of the disease. Accordingly, the preferred dose will be determined by a skilled medical doctor (for humans) or veterinary doctor or laboratory personnel (for non-human animals); however, typically the dose will lie between about 1 ng/kg to about 1000 mg/kg by weight of the patient or subject.

For use in the inventive method, the CAR agonist and/or PPAR antagonist compounds can be formulated for administration into the patient or subject by any desired route, such as parentarally (e.g., intravenous injection, intramuscular injection, subderman injection), orally (e.g., via immediate release or extended release tablets, capsules, lozenges, etc.), nasally or via oral inhalazion (e.g., using a nebulizer), transmucosally, via suppository, pessary, etc. It is within the ordinary skill to formulate such active compounds, together with conventional pharmaceutical excipients, for a desired route of administration. Additionally, the CAR agonist(s) and, if employed, PPAR antagonist(s) can be administered separately or in one or more (where more than two compounds are administered) combined formulations.

In accordance with the inventive method, by controlling obesity, the inventive method can improve body composition by decreasing fat mass and/or increasing lean mass. The invention can prevent and treat high fat-induced obesity and it can relieve genetically predisposed obesity. By controlling diabetes, the method can be employed as a treatment for high fat diet-induced type two diabetes as well as genetically predisposed diabetes.

In practicing the inventive method, it should be recognized that CAR is implicated in regulating drug-metabolizing enzymes. Accordingly, caution should be undertaken in patients and subjects taking other medications, as agonizing CAR might lead to drug-drug interactions.

The following examples further illustrate the invention but, of course, should not be construed as in any way limiting its scope. The following abbreviations are employed in these examples: LXR, liver X receptor; LXRE, LXR responsive element; PXR, pregnane X receptor; PBRE, phenobarbital response element; PPAR, peroxisome proliferator-activated receptor; CYP, cytochrome P450; Acc-1, acetyl CoA carboxylase 1; Fas, fatty acid synthase; Scd-1, stearoyl CoA desaturase-1; SRC1, steroid receptor co-activator 1; Srebp-1c, sterol regulatory element-binding protein 1c; Abcg⅝, ATP-binding cassette (ABC) transporters G5 and G8; Mrp2, multidrug resistance associated protein 2; FABP, fatty acid binding protein; TO1317 (T0901317), N-methyl-n-[4-(2,2,2-trifluoro-1-hydroxy-1-trifluoromethylethyl]-phenyThbenzenesulfonamide.

### EXAMPLE 1

This example demonstrates that agonizing CAR can control obesity.

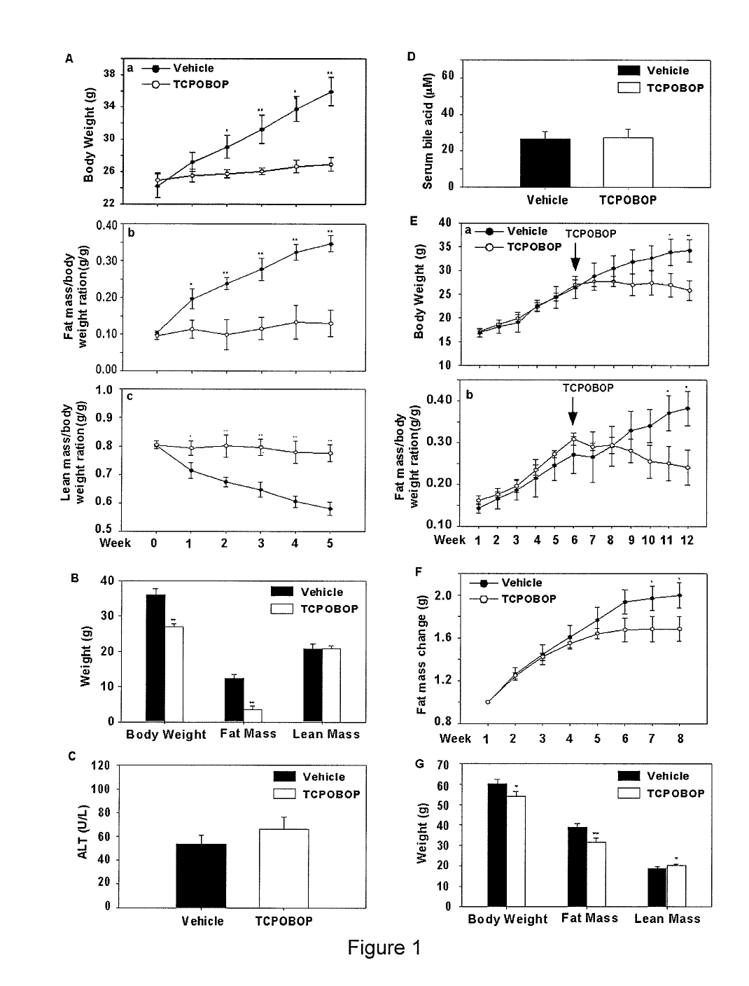

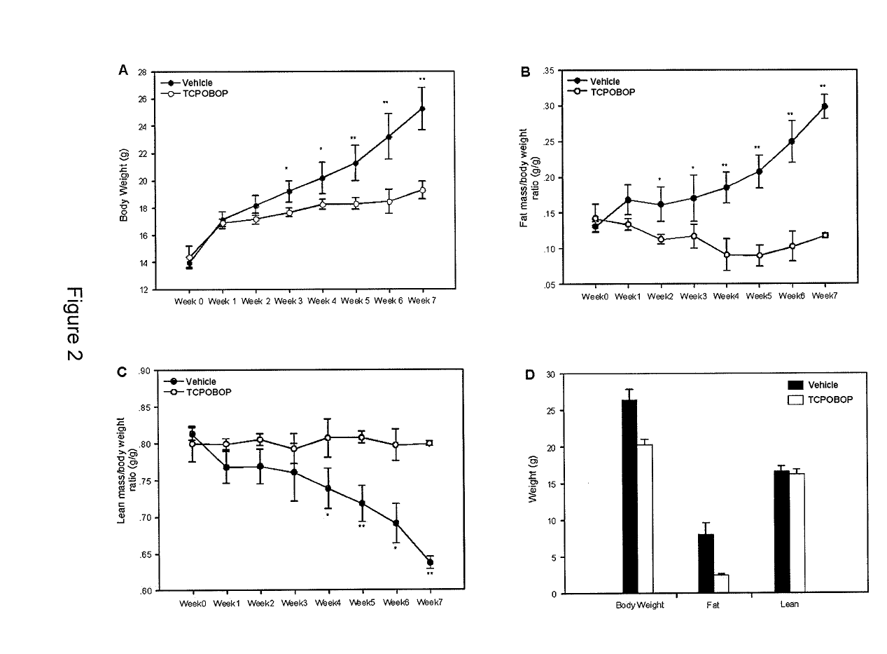

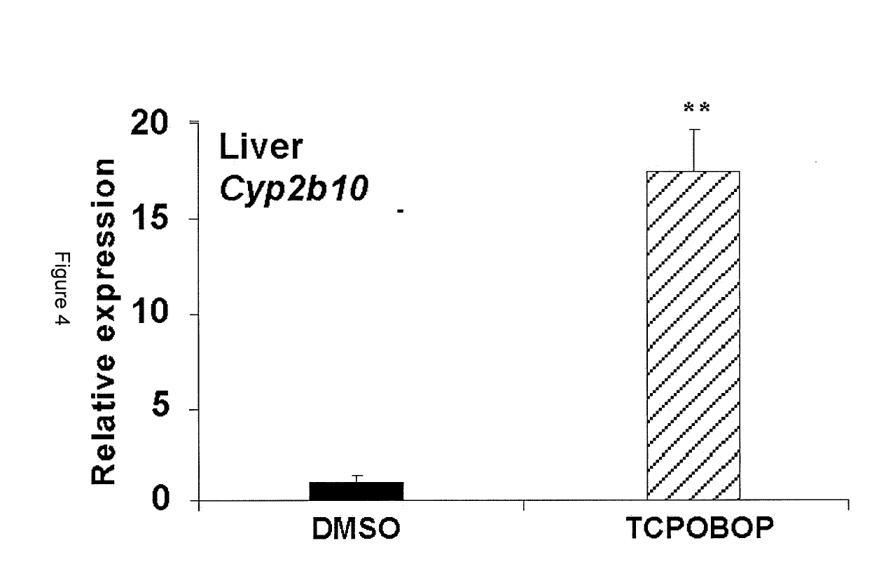

Eight-week male C57BL/6J mice were fed with high fat diet (HFD) for five weeks and simultaneously treated with the CAR agonist TCPOBOP (0.5 mg/kg, once a week) or vehicle (DMSO). As shown in FIG. 1A, TCPOBOP significantly inhibited gain of body weight after two weeks of drug treatment (FIG. 1A-a). MRI analysis showed that TCPOBOP significantly inhibited the gain of fat mass (FIG. 1A-b). The lean masses between the TCPOBOP and vehicle groups were not significantly different, and as such, the lean to body weight ratio decreased in vehicle-treated mice; whereas the ratio remained steady in the TCPOBOP group (FIG. 1A-c). The effect of TCPOBOP on the fat mass was so dramatic that the differences were obvious and significant after one week of drug treatment (FIGS. 1A-b and 1A-c). At the end of five-week treatment, the body weight differences between TCPOBOP group and vehicle group were completely accountable by the gain of fat mass in the TCPOBOP group (FIG. 1B). A similar pattern of TCPOBOP effect was observed in HFD-treated female C57BL/6J mice (FIG. 2), suggesting that the effect was not gender specific. The TCPOBOP effect was unlikely due to toxicity, because the two groups had similar serum levels of alanine aminotransferase (ALT) (FIG. 1C) and bile acid (FIG. 1D), both of which are indicators of hepatotoxicity. This regimen of TCPOBOP was sufficient to activate CAR, as evidenced by the activation of Cyp2b10, a prototypical CAR target gene, in the liver (FIG. 4).

To determine whether CAR activation is effective in controlling obesity in mice with pre-existing obesity, C57BL/6J female mice were fed with HFD for six weeks, followed by 6 weeks of TCPOBOP (0.5 mg/kg, once a week) treatment when mice remained on HFD. As shown in FIG. 1E, treatment with TCPOBOP stabilized the mouse body weight; whereas the vehicle group continued to gain body weight (FIG. 1E-a). The body weight of the TCPOBOP group was significantly lower than the vehicle group after 5 weeks of drug treatment. Again, the TCPOBOP effect on the body weight was largely attributed to the inhibition of gain of fat mass (FIG. 1E-b).

TCPOBOP was also effective in the genetically leptin deficient and obese ob/ob mice. In this experiment, 7-week old female ob/ob mice were maintained in chow diet and treated with TCPOBOP or vehicle for eight weeks. The TCPOBOP group had less gain of fat mass (FIG. 1F). By the end of eight week treatment, the TCPOBOP-treated ob/ob mice had significantly lower body weight compared to the vehicle group, which was accompanied by deceased fat mass and increased lean mass (FIG. 1G). Taken together, these data suggest that agonizing CAR has an anti-obesity effect.

### EXAMPLE 2

This example demonstrates that agonizing CAR can control diabetes.

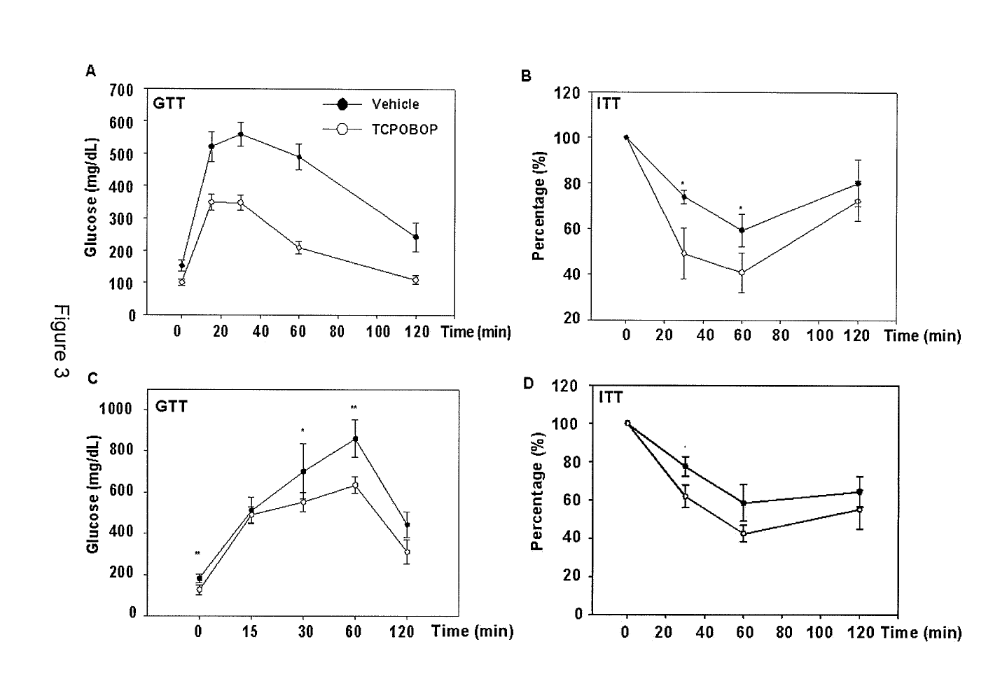

Since obesity is strongly associated with insulin resistance, a typical characteristic of type two diabetes, whether the CAR agonist can also improve insulin sensitivity was examined. Treatment of HFD-fed C57BL/6J male mice with TCPOBOP for five weeks significantly improved insulin sensitivity, as confirmed by both the glucose tolerance test (GTT) (FIG. 3A) and insulin tolerance test (ITT) (FIG. 3B). The insulin sensitizing effect of TCPOBOP was also evident in ob/ob mice (FIGS. 3C and 3D). When the serum chemistry was analyzed, it was found that treatment with TCPOBOP in HFD-fed C57BL/6J mice significantly decreased the levels of fasting glucose, insulin and triglyceride (Table 1). The cholesterol and free fatty acid levels also tended to be lower, but the differences did not reach statistical significance (Table 1). In the ob/ob mice, TCPOBOP treatment significantly decreased the levels of fasting glucose, serum triglyceride and serum cholesterol; whereas the changes in insulin and free fatty acid were not significant (Table 1).

TABLE 1
Fasting serum Glucose and lipid profile in TC treatment group v.s. Vehicle group

| | HFD-fed C57BL/6j mice | | ob/ob mice | |
|---|---|---|---|---|
| | Vehicle | TCPOBOP | Vehicle | TCPOBOP |
| Fasting Glucose (mg/dl) | 153 ± 18.3 | 102.8 ± 17.7** | 183.9 ± 21.3 | 128 .7 ± 23.4** |
| Insulin (ng/ml) | 3.26 ± 0.68 | 1.57 ± 0.37* | 17.89 ± 0.35 | 15.38.9 ± 1.85 |
| Total Triglyceride (mg/dl) | 230.2 ± 21.3 | 132.8 ± 17.7** | 162.5 ± 14.1 | 126.3 ± 13.7* |
| Total Cholesterol (mg/dl) | 100.8 ± 9.6 | 89.7 ± 5.3 | 118.4 ± 5.9 | 90.2 ± 9.9* |
| Free Fatty Acid (uM) | 8.4 ± 1.1 | 6.8 ± 0.6 | 25.9 ± 2.14 | 21.4 ± 1.54 |

### EXAMPLE 3

This example demonstrates that agonizing CAR can inhibit lipogenesis, hepatic VLDL secretion, and hepatic gluconeogenesis.

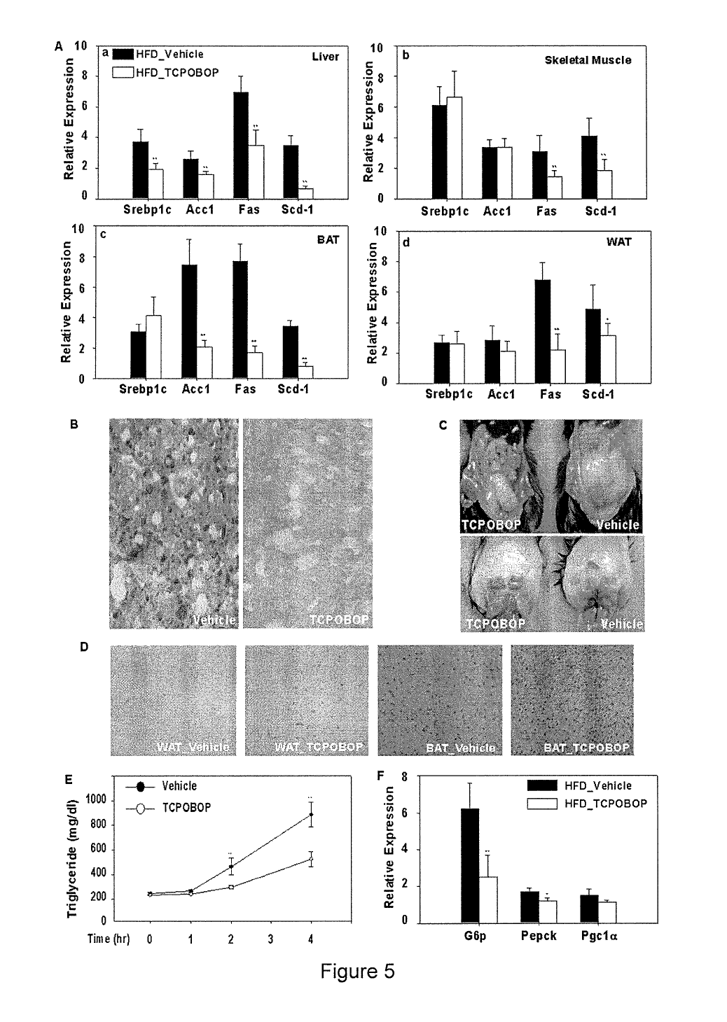

To understand the mechanisms by which CAR inhibits obesity and improves insulin sensitivity, gene expression was profiled in several relevant tissues, including the liver, skeletal muscle, white adipose tissue (WAT) and brown adipose tissue (BAT) in HFD-fed C57BL/6J mice treated with TCPOBOP for one week. In the liver, TCPOBOP suppressed the expression of lipogenic genes, including Srebp-1c, Acc-1, Fas and Scd-1 (FIG. 5A-a). Significant inhibition of Fas and Scd-1 gene expression was also seen in the skeletal muscle (FIG. 5A-b), BAT (FIG. 5A-c) and WAT (FIG. 5A-d) of TCPOBOP-treated mice. The expression of Acc-1 was also significantly inhibited by TCPOBOP in BAT (FIG. 5A-c). A similar pattern of gene regulation was observed in mice fed with HFD and treated with TCPOBOP for five weeks (data not shown). The inhibition of lipogenesis was further supported by the ameliorated hepatic steatosis (FIG. 5B), decreased sizes of abdomen WAT and BAT (FIG. 5C), and adipocyte hypotrophy (FIG. 5D) in HFD-fed mice treated with TCPOBOP for five weeks.

Since excessive VLDL production and triglyceride secretion contribute to the pathogenesis of hypertriglyceridemia, obesity and diabetes, VLDL secretion rate after a 5-week TCPOBOP treatment also was evaluated. As shown in FIG. 5E, VLDL secretion rate was significantly reduced in TCPOBOP group compared with vehicle group. The inhibition of VLDL secretion was consistent with the lower serum triglyceride level (data not shown) and inhibition of hepatic lipogenesis (FIG. 5A) in these animals. In the same groups of mice, the expression of two important gluconeogenic enzymes, glucose-6-phosphatase (G6p) and phosphoenolpyruvate carboxykinase (Pepck), was also significantly inhibited (FIG. 5F), consistent with the lower fasting glucose in these animals (Table 1).

### EXAMPLE 4

This example demonstrates that agonizing CAR can suppress lipogenic and gluconeogenic gene expression in a CAR-dependent manner.

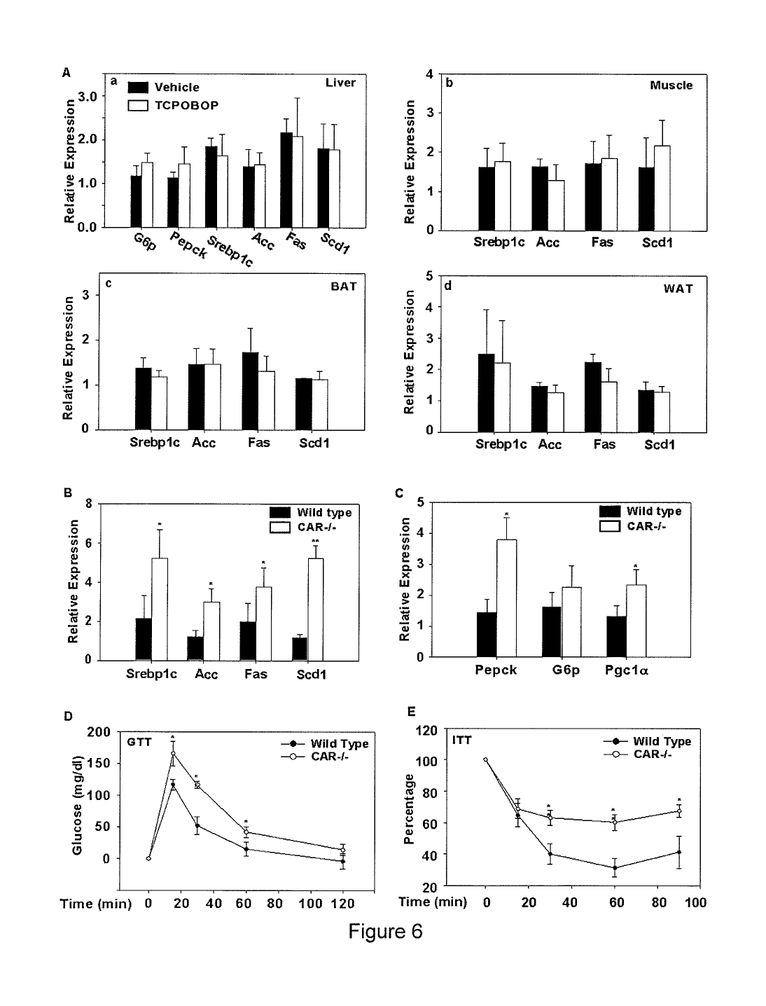

The suppression of lipogenic and gluconeogenic gene expression was also seen in TCPOBOP-treated wild type mice of C57BL/6J and SvJ129 mixed background (data not shown). To demonstrate whether the effect of TCPOBOP on the gene expression is CAR dependent, CAR^{-/-} mice of the C57BL/6J and SvJ129 mixed background were fed with HFD, in the presence or absence of TCPOBOP, for five week before tissue harvesting and gene expression analysis. As shown in FIG. 6A, the expression of none of the lipogenic and gluconeogenic genes was significantly altered by TCPOBOP in the liver (FIG. 6A-a), skeletal muscle (FIG. 6A-b), WAT (FIG. 6A-c), or BAT (FIG. 6A-d).

CAR is known to have a high basal transcriptional activity (Baes et al., Mol. Cell Biol.;14(3):1544-52 (1994)). Since activation of CAR suppressed the expression of lipogenic and gluconeogenic genes, it can be hyopthesized that the expression of the same genes may increase in CAR^{-/-} mice. Indeed, the expression of Srebp-1c, Scd-1, Fas, Acc-1 (FIG. 6B), Pepck and Pgc1α (FIG. 6C) was significantly elevated in CAR^{-/-} mice.

Consistent with the gene expression profiles, it was found that the chow-fed CAR^{-/-} mice showed significantly impaired insulin sensitivity when compared to their wild type littermates, as confirmed by GTT (FIG. 6D) and ITT (FIG. 6E) tests. Accordingly, the suppression of lipogenic and gluconeogenic gene expression by TCPOBOP is CAR dependent, and CAR^{-/-} mice show increased basal expression of lipogenic and gluconeogenic genes and are diabetic.

### EXAMPLE 5

This example demonstrates that agonizing CAR can increase BAT energy expenditure, decrease incomplete skeletal muscle β-oxidation, and induce lipid mobilization.

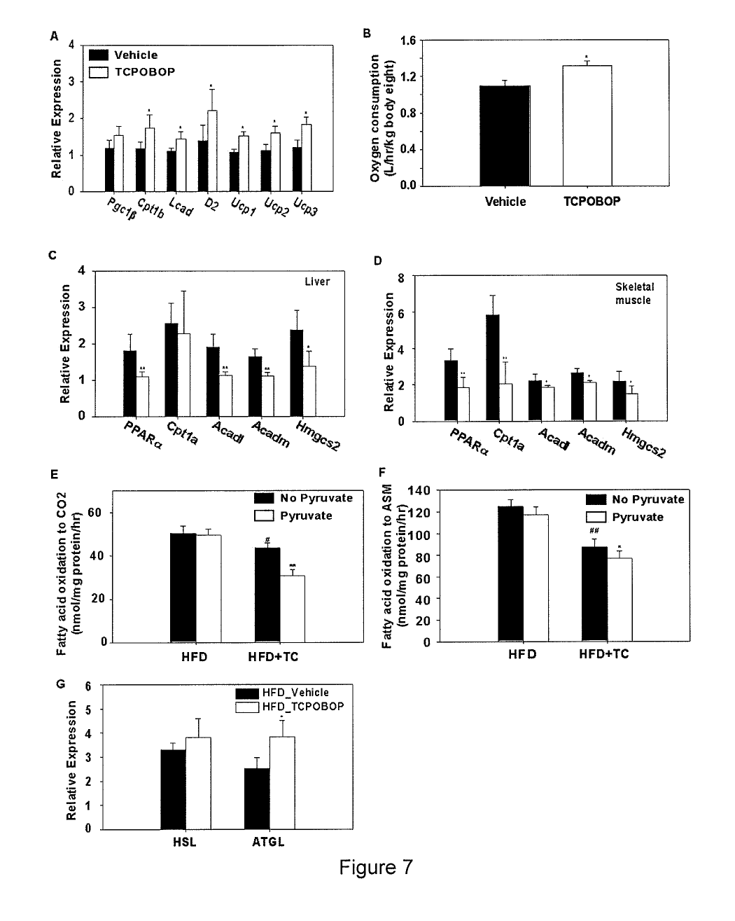

BAT plays an important role in regulating energy expenditure. The smaller BAT in TCPOBOP-treated mice prompted an examination of the expression of BAT genes involved in energy expenditure. As shown in FIG. 7A, TCPOBOP treatment significantly increased BAT expression of peroxisome proliferator activated receptor γ coactivator (PGC)-1b, muscle-type carnitine palmitoyltransferase I (mCPT-I), LCAD, D2, and uncoupling protein (UCP)-1, UCP-2 and UCP-3. Mice treated with TCPOBOP also had increased oxygen consumption (FIG. 7B), further supporting the augmented energy expenditure in TCPOBOP-treated mice.

The effect of TCPOBOP on the fatty acid oxidation in the liver and skeletal muscle also was investigated. Short-term (one week) TCPOBOP treatment had little effect on the expression of genes related to β-oxidation, except that expression of PPARα was modestly suppressed (data not shown). Surprisingly, chronic (five weeks) treatment with TCPOBOP significantly suppressed the expression of PPARα and its target genes involved in β-oxidation and ketogenesis in both the liver (FIG. 7C) and skeletal muscle (FIG. 7D). These include the suppression of long-chain acyl-CoA dehydrogenase (Aacdl), medium-chain acyl CoA dehydrogenase (Acadm) and HMG-CoA synthase (HMGCS2). When the mitochondrial β-oxidation rate was measured in the gastrocnemius muscle derived from HFD-fed mice, compared to the vehicle group, mice treated with TCPOBOP had a modest, although significant, decrease in the rate of [¹⁴C]-oleate oxidation to CO₂, an indicator of complete (β-oxidation (FIG. 7E). In contrast, TCPOBOP treatment resulted in a much more dramatic reduction in the accumulation of radiolabeled intermediates in the acid-soluble metabolite (ASM), an indicator of incomplete β-oxidation (FIG. 7F). These results suggest that TCPOBOP-treated mice had reduced incomplete β-oxidation. When pyruvate was added to the incubation buffer as a competing glucose-derived carbon source to induce substrate switch (Randle et al., Lancet.;1(7285):785-9 (1963)), mitochondria from vehicle-treated mice failed to exhibit a significant switch, which is suggestive of insulin resistance. In contrast, mitochondria from TCPOBOP-treated mice showed significant switch by having decreased CO₂ formation (FIG. 7E) and decreased incomplete β-oxidation (FIG. 7F), indicating improved insulin sensitivity.

Among other metabolic changes, the expression of adipose triglyceride lipase (ATGL), but not the hormone sensitive lipase (HSL), was induced in WAT of TCPOBOP-treated mice (FIG. 7G). These results suggest that activation of CAR promotes the lipid mobilization, and they are consistent with decreases in fat mass and adipocyte hypotrophy.

### EXAMPLE 6

This example demonstrates that agonizing CAR can decrease leptin production and inhibit food intake.

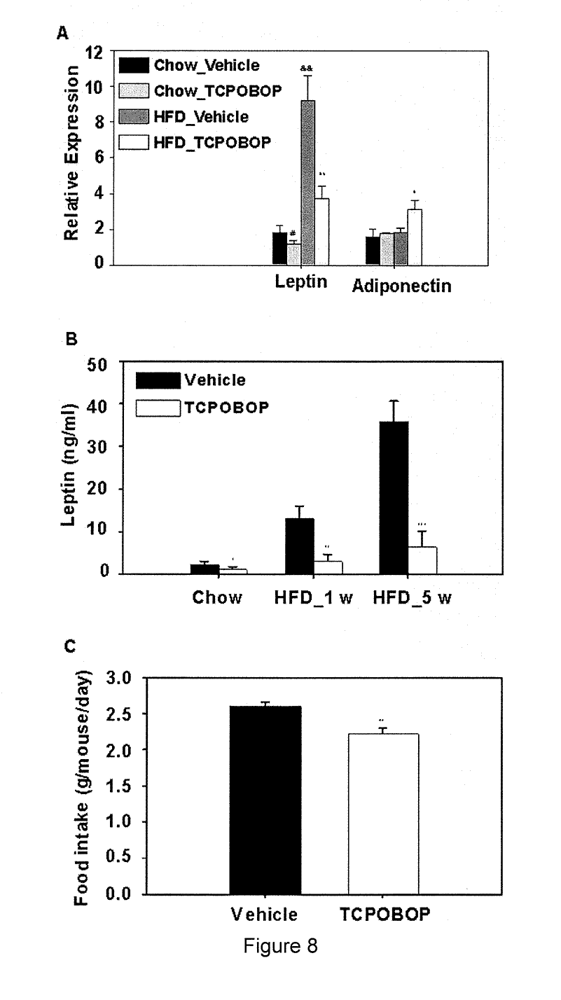

Leptin is an adipokine primarily produced in WAT. High fat diet has been shown to induce leptin mRNA level in WAT without inhibition of food intake, which indicates HFD-induced leptin resistance. HFD induced leptin mRNA expression as expected (FIG. 8A). However, the leptin mRNA level in TCPOBOP-treated and HFD-fed mice was dramatically lower than their vehicle-treated counterparts, which was accompanied by an increased expression of adiponectin (FIG. 8A). The decreased leptin production in TCPOBOP-treated mice was also confirmed at the protein level (FIG. 8B). Treatment with TCPOBOP also significantly reduced the food intake (FIG. 8C). The decreased production of leptin may have resulted from decreased fat mass, and the inhibition of food intake may have contributed to the anti-obesity effect.

### EXAMPLE 7

This example demonstrates that CAR is required for fasting glucose metabolism adaptation.

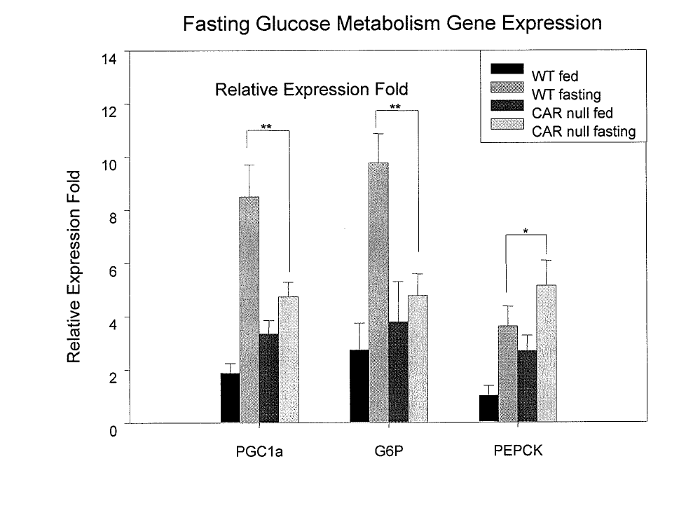

In wild type mice, it is known that in response to overnight fasting, the expression of PGC1a, G6P and PEPCK will increase. G6P and PEPCK are two essential enzymes for gluconeogenesis. PGC1a is a nuclear receptor co-activator known to play a positive role in the regulation of G6P and PEPCK. In CAR null mice, the fasting glucose response is defective as compared to the response in wild-type mice. Specifically, as indicated in FIG. 9, the responses of PGC1 a and G6P appear to be completely abolished in CAR-null mice.

### EXAMPLE 8

This example demonstrates that CAR over-expression in liver decreases both fed and fasting glucose level.

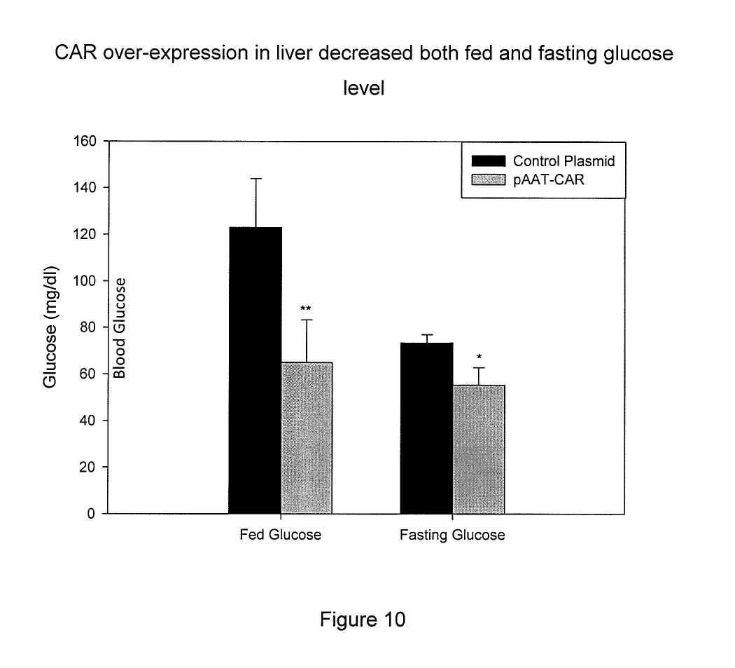

The livers of wild type mice were transfected with CAR by using the hydrodynamic gene delivery method. The results (FIG. 10) show that overexpression of CAR can significantly lower the blood glucose levels in both the fast and fed states. This result is consistent with the anti-diabetic effect of CAR discussed in other Examples herein.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of controlling obesity in a mammalian patient or subject in need thereof, comprising agonizing the constitutive androstane receptor (CAR) within the patient or subject.

2. The method of claim 1, which comprises not agonizing a peroxisome proliferator-activated receptor (PPAR) within the patient or subject, wherein the PPAR is selected from the group of PPARs consisting of PPARα, PPARβ/δ, PPARγ, and a combination of two or more thereof.

3. A method of controlling type two diabetes in a patient or subject in need thereof, comprising agonizing CAR within the human without agonizing a PPAR within the patient or subject, wherein the PPAR is selected from the group of PPARs consisting of PPARα, PPARβ/δ, PPARγ, and a combination of two or more thereof.

4. The method of claim 1, wherein CAR is agonized by administering to the patient or subject a CAR agonist in an amount and at a location to agonize CAR within the patient or subject.

5. The method of claim 4, wherein the CAR agonist is selected from the group of agonists consisting of 1,4-bis[2-(3,5 dichloropyridyloxy)] benzene (TCPOBOP), Phenobarbital, 6-(4-chlorophenyl)imidazo[2,1-b][1,3]thiazole-5-carbaldehyde O-(3,4-dichlorobenzyl)oxime (CITCO), 6,7-Dimethylesculetin, oltipraz (OPZ), a pharmaceutically acceptable salt or hydrate of any thereof, and a combination of 2 or more thereof.

6. The method of claim 4, wherein the CAR agonist is administered parenterally.

7. The method of claim 6, wherein the CAR agonist is administered via intravenous injection.

8. The method of claim 4 wherein the CAR agonist is administered orally.

9. The method of claim 1, wherein CAR is agonized by administering a decoction of Artemisia capillaris to the patient or subject.

10. The method of claim 9, wherein the Artemisia capillaris is Yin Chin.

11. The method of claim 9, wherein the decoction is Yin Zhi Huang.

12. The method of claim 1, wherein CAR is agonized by increasing the expression of a CAR gene within the patient or subject.

13. The method of claim 12, wherein the expression is increased by introducing into the patient or subject a CAR expression vector under conditions for the CAR sequence within the vector to be expressed within the patient or subject to produce CAR within the patient or subject.

14. The method of claim 1, comprising antagonizing a PPAR within the patient or subject, wherein the PPAR is selected from the group of PPARs consisting of PPARα, PPARβ/δ, PPARγ, and a combination of two or more thereof.

15. The method of claim 14, wherein the PPAR is antagonized by administering to the patient or subject a PPAR antagonist in an amount and at a location to antagonize the PPAR within the patient or subject.

16. The method of claim 15, wherein the PPAR antagonist is [(2S)-2-[[(1Z)-1-Methyl-3-oxo-[4-(trifluoromethyl)phe nyl]-1-propenyl]amino]-3-[4-[2-(5-methyl-2-phenyl-4-oxa zolyl)ethoxy]phenyl]propyl]carbamic acid ethyl ester.

17. The method of claim 1, wherein the patient or subject is human.

18. The method of claim 17, with the proviso that if a CAR agonist is administered to the human patient or subject, the agonist does not comprise TCPOBOP.
